(19) United States
(12) Patent Application Publication
(10) Pub. No.: US 2026/0301882 A1
(43) Pub. Date: Oct. 1, 2026

(54) ARTIFICIAL INTELLIGENCE-BASED SYSTEM FOR GENERATING DRUG LIKE MOLECULES AND METHOD THEREOF

(71) Applicant: CENTELLA SCIENTIFIC PRIVATE LIMITED, HYDERABAD (IN)

(72) Inventors: RIYAZ SYED, HYDERABAD (IN); POORNA CHANDRA RAO YEDLA, HYDERABAD (IN)

(21) Appl. No.: 19/231,643

(22) Filed: Jun. 9, 2025

(30) Foreign Application Priority Data
Mar. 25, 2025 (IN) 202541027886

(51) Int. Cl. G16C 20/50; G16C 20/30; G16C 20/70

(52) U.S. Cl. CPC G16C 20/50; G16C 20/30; G16C 20/70

(57) ABSTRACT

The present invention discloses an artificial intelligence-based (AI-based) system for generating drug like molecules and an artificial intelligence-based (AI-based) method thereof. The AI-based system obtains biological target data and therapeutic information. The AI-based system generates molecular structures. The AI-based system evaluates the generated molecular structures for binding affinity, pharmacological relevance, and structural validity to compute a multi-parametric scoring. The AI-based system predicts properties of the evaluated molecular structures. The AI-based system determines feasible synthesis pathways for the selected molecular structures. The AI-based system optimises the molecular structures for generating the drug like molecules with validated synthetic feasibility and drug-like properties.

### EARLIEST PRIORITY DATE

This application claims priority from a Provisional patent application filed in India having patent application No. 202541027886, filed on 25 Mar. 2025 and titled “ARTIFICIAL INTELLIGENCE-BASED SYSTEM FOR GENERATING DRUG LIKE MOLECULES AND METHOD THEREOF”.

### FIELD OF INVENTION

Embodiments of the present invention relate to drug discovery systems and more particularly relate to an artificial intelligence-based (AI-based) system for generating one or more drug like molecules and an artificial intelligence-based (AI-based) method thereof.

### BACKGROUND

A field of drug discovery has evolved significantly in recent years, with computational methods and artificial intelligence (AI) technologies playing an increasingly prominent role. The computational methods and AI technologies aim to enhance efficiency in identifying and developing new therapeutic compounds. Computer-aided drug discovery (CADD) has emerged as a promising area, leveraging computational resources to accelerate various stages of a drug development pipeline.

Traditional drug discovery workflows involve iterative Design-Make-Test-Analyse (DMTA) cycles, which may be resource-intensive. The DTMA cycles include multiple rounds of molecular construction, synthesis, and experimental testing, potentially leading to extended timelines and increased costs. The complexity of biological systems and a vast chemical space present ongoing challenges in identifying viable one or more drug like molecules.

Recent advancements in machine learning and deep learning techniques have introduced new possibilities for enhancing drug discovery processes. Generative models have shown potential in creating novel one or more molecular structures. Virtual screening methods have evolved to handle larger compound libraries, while improvements in property prediction models have enhanced the ability to estimate characteristics of the one or more molecular structures. However, despite these advancements, challenges remain in fully integrating these technologies into the drug discovery pipeline, particularly in terms of ensuring the accuracy of predictions, handling the complexity of the biological systems, and maintaining efficiency across large-scale datasets.

Several limitations persist in current CADD approaches. Many existing platforms provide fragmented solutions, focusing on individual aspects of the drug discovery process without providing seamless integration across different stages. This fragmentation may affect data transfer and workflow management. Additionally, the accuracy of predictions for complex properties, such as absorption, distribution, metabolism, excretion, and toxicity (ADMET), remains an area of ongoing research, particularly for novel chemical entities.

Retrosynthesis planning, an integral part of drug discovery, has benefited from computational approaches but still faces challenges in predicting one or more feasible synthesis pathways for novel compounds. The incorporation of reaction condition predictions and reagent availability considerations into retrosynthesis models represents an ongoing area of development.

In an existing technology, a system for generating the one or more molecular structures using one or more machine learning models is disclosed. The system includes a molecular generator that uses a variational autoencoder to create the one or more molecular structures based on input data. While the system addresses aspects of molecular generation, the system may not fully integrate other components of the drug discovery pipeline, such as virtual screening and retrosynthesis planning, into a comprehensive platform.

As the field of AI-driven drug discovery continues to evolve, there is a growing interest in integrated platforms that may effectively combine various computational techniques and leverage large-scale data resources. Such platforms may potentially address the limitations of current approaches and provide more efficient tools for identifying promising one or more drug like molecules. Therefore, there is a need for an integrated computational platform that addresses the limitations of current approaches in AI-driven drug discovery.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In order to overcome the above deficiencies of the prior art, the present disclosure is to solve the technical problem by providing an artificial intelligence-based (AI-based) method for generating one or more drug like molecules.

In accordance with an embodiment of the present invention, the AI-based method for generating the one or more drug like molecules is disclosed. In the first step, the AI-based method includes obtaining, by one or more hardware processors through a data-obtaining subsystem, one of: biological target data and therapeutic information from a user. The biological target data comprises at least one of: protein structures, ligand data, gene expression profiles, biomolecular interaction networks, and disease-associated biomarkers. The therapeutic information comprises at least one of: specific pharmacological profiles, target disease pathways, mechanism of action data, bioactivity annotations, and known drug resistance profiles.

In the next step, the AI-based method includes generating, by the one or more hardware processors through a generative molecule devising subsystem, one or more molecular structures through one or more machine learning (ML) models. The one or more ML models are configured to perform at least one of: binding affinity optimization, structural conformity and feasibility, chemical space exploration, and iterative feedback loop based on the obtained one of: biological target data and therapeutic information. The AI-based method further includes employing, by the one or more ML models, one of: one or more structure-based approaches and ligand-based procedures to generate a pharmacophore-based configuration associated with the one or more molecular structures to analyse at least one of: one or more molecular graphs and generate precise three dimensional (3D) conformers. The AI-based method further includes generating, by the one or more ML models, the one or more molecular structures based on incorporating at least one of: a binding site analysis and structure-based design principles, to optimise binding interactions with a biological target. The AI-based method further includes providing, by the one or more ML models, a structural conformity by generating the one or more molecular structures matches with a binding site geometry and maintaining synthetic feasibility. The one or more ML models comprise at least one of: one or more reinforcement learning-based (RL) models, one or more transformer-based architectures, one or more protein and chemical language models, one or more Large Language Models (LLMs), one or more Small Large Language Models (SLLMs), one or more Graphics Processing Unit (GPU)-based models, and one or more quantum models, to generate the one or more molecular structures with optimised binding affinity.

In the next step, the AI-based method includes evaluating, by the one or more hardware processors through a virtual screening subsystem, the generated one or more molecular structures for at least one of: binding affinity, pharmacological relevance, and structural validity via one or more virtual screening models to compute a multi-parametric scoring. The AI-based method further includes ranking, by the virtual screening subsystem, the generated one or more molecular structures based on the multi-parametric scoring. The multi-parametric scoring comprises at least one of: docking score, ligand efficiency score, and pharmacophore match score. The one or more virtual screening models comprise at least one of: computational docking models and quantitative structure-activity relationship (QSAR) models.

In the next step, the AI-based method includes predicting, by the one or more hardware processors through a prediction subsystem, one or more properties associated with absorption, distribution, metabolism, excretion, and toxicity (ADMET) of the evaluated one or more molecular structures by utilising one or more generative artificial intelligence (AI) models trained on diverse datasets consisting at least one of: pharmacokinetic data and toxicology data. The one or more properties comprise at least one of: solubility, permeability, metabolic stability, and potential toxicity. The one or more generative AI models are one or more graph neural network (GNN)-based models. Training the one or more GNN-based models on the diverse datasets includes at least one of: the pharmacokinetic data and the toxicology data to predict essential drug-like properties.

In the next step, the AI-based method includes determining, by the one or more hardware processors through a retrosynthesis planning subsystem, one or more feasible synthesis pathways for the selected one or more molecular structures through one or more retrosynthesis models based on at least one of: analysis reaction pathways and integration of one or more chemical reaction databases enabled by reaction informatics. The one or more retrosynthesis models comprise at least one of: one or more reaction template-based models and one or more transformer-based retrosynthesis models. The one or more retrosynthesis models employ at least one of: single-step reactions procedures and multi-step synthesis pathway procedures to determine the one or more feasible synthesis pathways along with at least one of: optimal reaction conditions, experimental procedures, impurity and yield predictions to report practical lab implementations. The AI-based method further includes generating, by the retrosynthesis planning subsystem, the one or more feasible synthesis pathways by integrating vendor-supplied reagent availability data to provide a streamlined synthesis planning.

In the next step, the AI-based method includes optimising, by the one or more hardware processors through an iterative refinement subsystem, the one or more molecular structures to generate the one or more drug like molecules with a validated synthetic feasibility and drug-like properties based on one or more feedback from at least one of: the generative molecule devising subsystem, the virtual screening subsystem, the prediction subsystem, and the retrosynthesis planning subsystem. The AI-based method further includes incorporating, by the iterative refinement subsystem, multi-objective optimization procedures to balance at least one of: binding affinity, toxicity, synthetic accessibility, bioavailability, and other related parameters in generating the one or more drug like molecules.

The AI-based method further includes collecting, by the one or more hardware processors through a data collection subsystem, curated datasets comprise at least one of: protein structures, chemical libraries, reaction pathways, and absorption, distribution, metabolism, excretion, and toxicity (ADMET) profiles. The AI-based method further includes pre-processing, by the one or more hardware processors through a data pre-processing subsystem, the curated datasets into a defined format by performing at least one of: eliminating duplicates and annotating relevant one or more features comprise at least one of: pharmacophores and binding affinities.

The AI-based method further includes training, by the one or more hardware processors through a model training subsystem, at least one of: the one or more ML models and the one or more generative AI models, on diverse chemical datasets for structure-based molecule representations, ligand-based molecule structures, and bioactivity-labelled datasets. The AI-based method further includes devising, by the one or more hardware processors through the model training subsystem, the one or more virtual screening models based on leveraging at least one of: one or more ligand-protein interactions, quantitative structure-activity relationship (QSAR) analysis, and docking procedures. The AI-based method further includes creating, by the one or more hardware processors through the model training subsystem, the one or more retrosynthesis models by incorporating at least one of: reaction templates and text-based pathways.

In accordance with an embodiment of the present invention, an artificial intelligence-based (AI-based) system for generating the one or more drug like molecules is disclosed. The AI-based system comprises one or more servers. The one or more servers comprises the one or more hardware processors and a memory unit. The memory unit is coupled to the one or more hardware processors, wherein the memory comprises a plurality of subsystems in form of one or more instructions executable by the one or more hardware processors. The plurality of subsystems comprises the data-obtaining subsystem, the generative molecule devising subsystem, the virtual screening subsystem, the prediction subsystem, the retrosynthesis planning subsystem, and the iterative refinement subsystem.

Yet in another embodiment, the data-obtaining subsystem is configured to obtain one of: the biological target data and the therapeutic information from the user. Yet in another embodiment, the generative molecule devising subsystem is configured to generate the one or more molecular structures through the one or more ML models. The one or more ML models are configured to perform at least one of: the binding affinity optimization, the structural conformity and feasibility, the chemical space exploration, and the iterative feedback loop based on the obtained one of: biological target data and therapeutic information.

Yet in another embodiment, the virtual screening subsystem is configured to evaluate the generated one or more molecular structures for at least one of: the binding affinity, the pharmacological relevance, and the structural validity via the one or more virtual screening models to compute the multi-parametric scoring. Yet in another embodiment, the prediction subsystem is configured to predict the one or more properties associated with the ADMET of the evaluated one or more molecular structures by the one or more generative AI models trained on the diverse datasets consisting at least one of: the pharmacokinetic data and the toxicology data.

Yet in another embodiment, the retrosynthesis planning subsystem is configured to determine the one or more feasible synthesis pathways for the selected one or more molecular structures through the one or more retrosynthesis models based on at least one of: the analysing reaction pathways and the integration of one or more chemical reaction databases enabled by the reaction informatics.

Yet in another embodiment, the iterative refinement subsystem is configured to optimise the one or more molecular structures for generating the one or more drug like molecules with the validated synthetic feasibility and the drug-like properties based on the one or more feedback from at least one of: the generative molecule devising subsystem, the virtual screening subsystem, the prediction subsystem, and the retrosynthesis planning subsystem.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE DRAWINGS

The disclosure will be described and explained with additional specificity and detail with the accompanying figures in which:

FIG. 1 illustrates an exemplary block diagram representation of a network architecture depicting an artificial intelligence-based (AI-based) system for generating one or more drug like molecules, in accordance with an embodiment of the present disclosure;

FIG. 2A illustrates an exemplary block diagram representation of the AI-based system as shown in FIG. 1 for generating the one or more drug like molecules, in accordance with an embodiment of the present disclosure;

FIG. 2B illustrates an exemplary block diagram depicting the AI-based system for generating the one or more drug like molecules, in accordance with an embodiment of the present disclosure;

FIG. 2C illustrates an exemplary visual representation depicting an output of the AI-based system, in accordance with an embodiment of the present disclosure; and

FIG. 3 illustrates an exemplary flow diagram depicting an artificial intelligence-based (AI-based) method for generating the one or more drug like molecules, in accordance with an embodiment of the present disclosure.

Further, those skilled in the art will appreciate that elements in the figures are illustrated for simplicity and may not have necessarily been drawn to scale. Furthermore, in terms of the method steps, chemical compounds, equipment and parameters used herein may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE PRESENT INVENTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more components, compounds, and ingredients preceded by “comprises . . . a” does not, without more constraints, preclude the existence of other components or compounds or ingredients or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present invention relate to an artificial intelligence-based (AI-based) system for generating one or more drug like molecules.

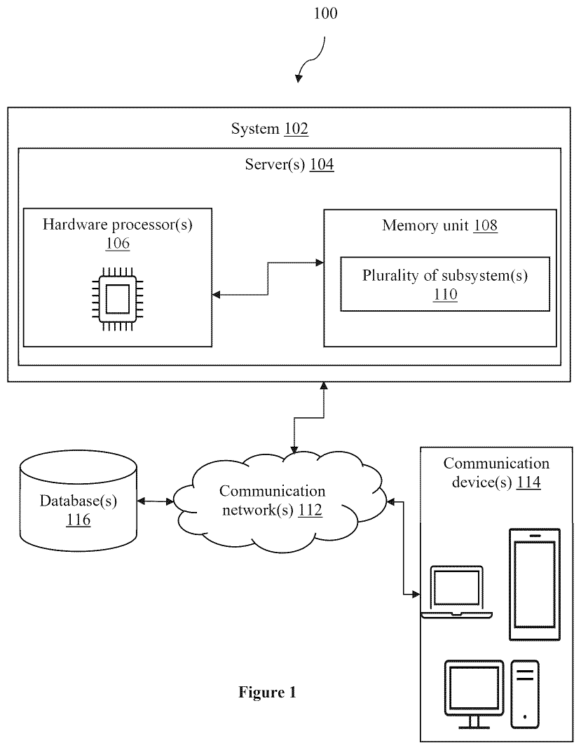

FIG. 1 illustrates an exemplary block diagram representation of a network architecture 100 depicting the AI-based system 102 for generating the one or more drug like molecules, in accordance with an embodiment of the present disclosure.

According to an exemplary embodiment of the disclosure, the AI-based system 102 (hereinafter referred to as the system 102) for generating the one or more drug like molecules is disclosed. The network architecture 100 may include the system 102, one or more databases 116, and one or more communication devices 114. The system 102, the one or more databases 116, and the one or more communication devices 114 may be communicatively coupled via one or more communication networks 112, ensuring seamless data transmission, processing, and decision-making. The system 102 acts as a central processing unit within the network architecture 100, responsible for generating the one or more drug like molecules.

In an exemplary embodiment, the system 102 comprises one or more servers 104. The one or more servers 104 may comprise a combination of discrete components, an integrated circuit, an application-specific integrated circuit, a field-programmable gate array, a digital signal processor, or other suitable hardware. The one or more servers 104 comprises one or more hardware processors 106 and a memory unit 108. The memory unit 108 is operatively connected to the one or more hardware processors 106. The memory unit 108 comprises one or more instructions in the form of a plurality of subsystems 110, configured to be executed by the one or more hardware processors 106.

In an exemplary embodiment, the one or more hardware processors 106 may include, for example, microprocessors, microcomputers, microcontrollers, digital signal processors, central processing units, state machines, logic circuits, and/or any devices that manipulate data or signals based on operational instructions. Among other capabilities, the one or more hardware processors 106 may fetch and execute the one or more instructions in the memory unit 108 operationally coupled with the system 102 for performing tasks such as data processing, input/output processing, and/or any other functions. Any reference to a task in the present disclosure may refer to an operation being or that may be performed on data. The one or more hardware processors 106 are high-performance processors capable of handling large volumes of data and complex computations. The one or more hardware processors 106 may be, but not limited to, at least one of: multi-core central processing units (CPU), graphics processing units (GPUs), and the like, which enhance an ability of the system 102 to process real-time data from one or more sources simultaneously.

In an exemplary embodiment, the one or more databases 116 may configured to store and manage data related to various aspects of the system 102. The one or more databases 116 may store data associated with at least one of, but not limited to, the one or more drug like molecules, one or more machine learning (ML) models, one or more generative artificial intelligence (AI) models, any other information necessary for the functionality and optimization of the system 102, and the like. The one or more databases 116 may include different types of databases such as, but not limited to, relational databases (e.g., Structured Query Language (SQL) databases such as PostgresDB and Oracle® databases), non-Structured Query Language (NoSQL) databases (e.g., MongoDB, Cassandra), time-series databases (e.g., InfluxDB), an OpenSearch database, object storage systems (e.g., Amazon® S3), and the like.

In an exemplary embodiment, the one or more communication devices 114 are configured to enable one or more users to interact with the system 102. The one or more communication devices 114 may be digital devices, computing devices, and/or networks. The one or more communication devices 114 may include, but not limited to, a mobile device, a smartphone, a personal digital assistant (PDA), a tablet computer, a phablet computer, a wearable computing device, a virtual reality/augmented reality (VR/AR) device, a laptop, a desktop, and the like.

In an exemplary embodiment, the one or more communication networks 112 may be, but not limited to, a wired communication network and/or a wireless communication network, a local area network (LAN), a wide area network (WAN), a Wireless Local Area Network (WLAN), a metropolitan area network (MAN), a telephone network, such as the Public Switched Telephone Network (PSTN) or a cellular network, an intranet, the Internet, a fibre optic network, a satellite network, a cloud computing network, a combination of networks, and the like. The wired communication network may comprise, but not limited to, at least one of: Ethernet connections, Fiber Optics, Power Line Communications (PLCs), Serial Communications, Coaxial Cables, Quantum Communication, Advanced Fiber Optics, Hybrid Networks, and the like. The wireless communication network may comprise, but not limited to, at least one of: wireless fidelity (wi-fi), cellular networks (including fourth generation (4G) technologies and fifth generation (5G) technologies), Bluetooth®, ZigBee®, long-range wide area network (LoRaWAN), satellite communication, radio frequency identification (RFID), 6G (sixth generation) networks, advanced IoT protocols, mesh networks, non-terrestrial networks (NTNs), near field communication (NFC), and the like.

In an exemplary embodiment, the system 102 may be implemented by way of a single device or a combination of multiple devices that may be operatively connected or networked together. The system 102 may be implemented in hardware or a suitable combination of hardware and software.

Though few components and the plurality of subsystems 110 are disclosed in FIG. 1, there may be additional components and subsystems which is not shown, such as, but not limited to, ports, routers, repeaters, firewall devices, network devices, the one or more databases 116, network attached storage devices, assets, machinery, instruments, facility equipment, emergency management devices, image capturing devices, any other devices, and combination thereof. The person skilled in the art should not be limiting the components/subsystems shown in FIG. 1. Although FIG. 1 illustrates the system 102, and the one or more communication devices 114 connected to the one or more databases 116, one skilled in the art can envision that the system 102, and the one or more communication devices 114 may be connected to several user devices located at various locations and several databases via the one or more communication networks 112.

Those of ordinary skilled in the art will appreciate that the hardware depicted in FIG. 1 may vary for particular implementations. For example, other peripheral devices such as an optical disk drive and the like, the local area network (LAN), the wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognize that, for simplicity and clarity, the full structure and operation of all data processing systems suitable for use with the present disclosure are not being depicted or described herein. Instead, only so much of the system 102 as is unique to the present disclosure or necessary for an understanding of the present disclosure is depicted and described. The remainder of the construction and operation of the system 102 may conform to any of the various current implementations and practices that were known in the art.

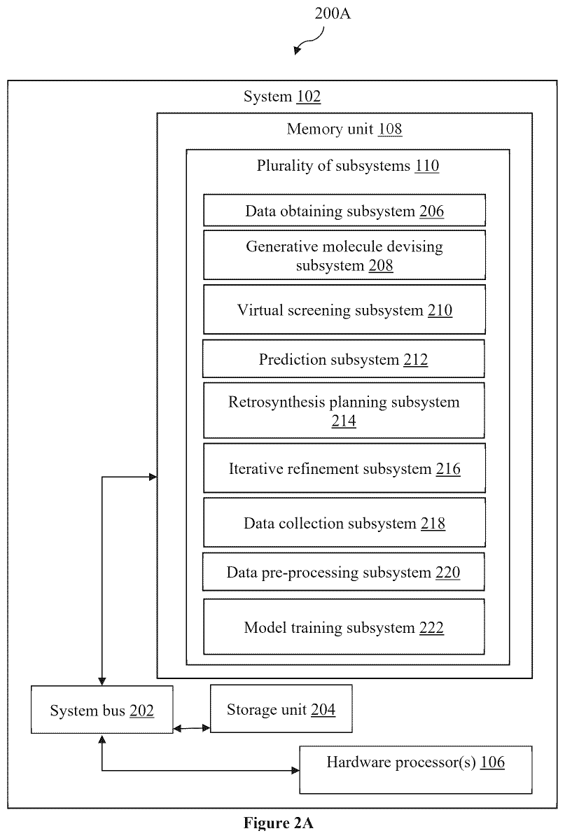

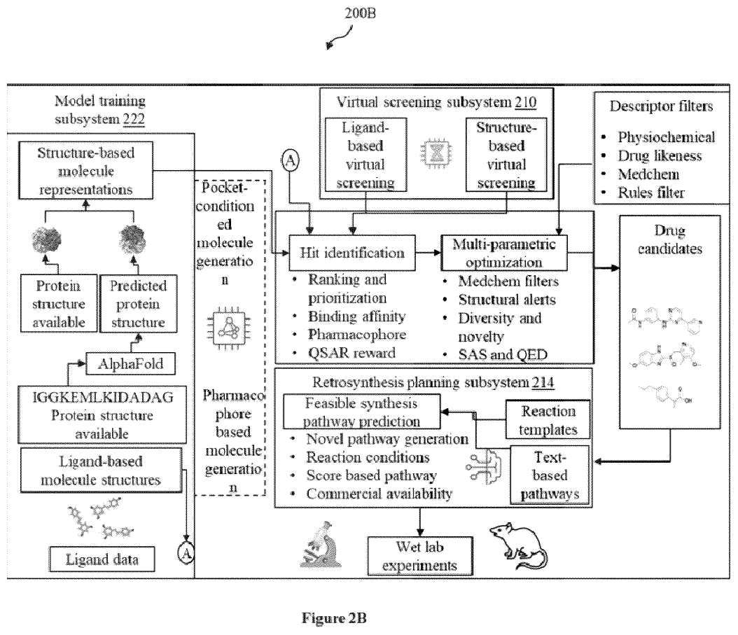

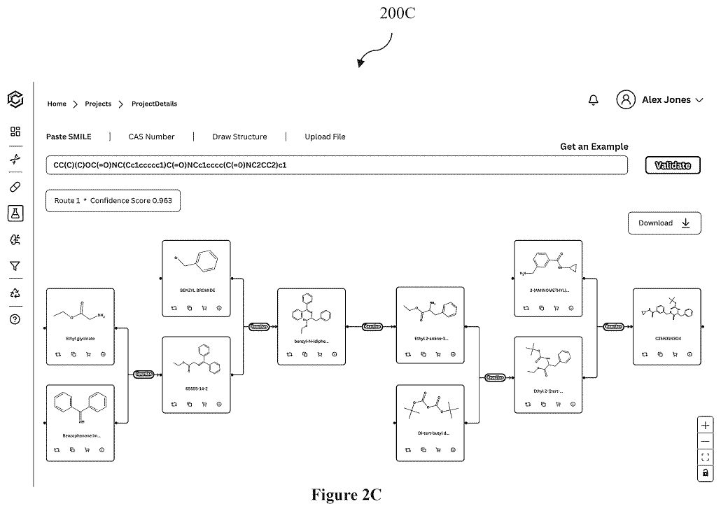

FIG. 2A illustrates an exemplary block diagram representation 200A of the system 102 as shown in FIG. 1 for generating the one or more drug like molecules, in accordance with an embodiment of the present disclosure;

FIG. 2B illustrates an exemplary block diagram 200B depicting the system 102 for generating the one or more drug like molecules, in accordance with an embodiment of the present disclosure; and

FIG. 2C illustrates an exemplary visual representation 200C depicting an output of the system 102, in accordance with an embodiment of the present disclosure.

In an exemplary embodiment, the system 102 comprises the one or more servers 104, the memory unit 108, and a storage unit 204. The one or more hardware processors 106, the memory unit 108, and the storage unit 204 are communicatively coupled through a system bus 202 or any similar mechanism. The system bus 202 functions as a central conduit for data transfer and communication between the one or more hardware processors 106, the memory unit 108, and the storage unit 204. The system bus 202 facilitates the efficient exchange of information and instructions, enabling the coordinated operation of the system 102.

In an exemplary embodiment, the memory unit 108 is operatively connected to the one or more hardware processors 106. The memory unit 108 comprises the plurality of subsystems 110 in the form of the one or more instructions executable by the one or more hardware processors 106. The plurality of subsystems 110 comprises a data-obtaining subsystem 206, a generative molecule devising subsystem 208, a virtual screening subsystem 210, a prediction subsystem 212, a retrosynthesis planning subsystem 214, an iterative refinement subsystem 216, a data collection subsystem 218, a data pre-processing subsystem 220, and a model training subsystem 222. The one or more hardware processors 106 associated within the one or more servers 104, as used herein, means any type of computational circuit, such as, but not limited to, the microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 106 may also include embedded controllers, such as generic or programmable logic devices or arrays, application-specific integrated circuits, single-chip computers, and the like.

The memory unit 108 may be the non-transitory volatile memory unit and the non-volatile memory unit. The memory unit 108 may be coupled to communicate with the one or more hardware processors 106, such as being a computer-readable storage medium. The memory unit 108 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the memory unit 108 includes the plurality of subsystems 110 stored in the form of the one or more instructions on any of the above-mentioned storage media and may be in communication with and executed by the one or more hardware processors 106.

The storage unit 204 may be a cloud storage or the one or more databases 116 such as those shown in FIG. 1. The storage unit 204 may store, but not limited to, recommended course of action sequences dynamically generated by the system 102. The action sequences comprise data obtaining, generative molecule devising, virtual screening, prediction, retrosynthesis planning, iterative refinement, data collection, data pre-processing, model training, and the like. Additionally, the storage unit 204 may retain previous action sequences for comparison and future reference, enabling continuous refinement of the system 102 over time. The storage unit 204 may be any kind of database such as, but not limited to, relational databases, dedicated databases, dynamic databases, monetized databases, scalable databases, cloud databases, distributed databases, any other databases, and a combination thereof.

In an exemplary embodiment, the data-obtaining subsystem 206 is configured to obtain at least one of: biological target data and therapeutic information from a user of the one or more users for advanced biomedical research and personalized medicine applications. The biological target data may comprise, but not limited to, at least one of: protein structures, ligand data, gene expression profiles, biomolecular interaction networks, disease-associated biomarkers, and the like. The protein structures provide insights into three-dimension (3D) configuration of target one or more molecular structures, which is essential for drug binding and therapeutic intervention. The ligand data includes details of small one or more molecular structures that interact with proteins, aiding in drug discovery and optimization. The gene expression profiles provide crucial information on how genes are activated and suppressed in different conditions, assisting in the identification of potential therapeutic targets. The biomolecular interaction networks map the relationships between various biological molecules, enabling the understanding of complex cellular pathways. The disease-associated biomarkers serve as indicators of pathological conditions, facilitating early diagnosis and precision treatment strategies.

The therapeutic information may comprise, but not constrained to, at least one of: specific pharmacological profiles, target disease pathways, mechanism of action data, bioactivity annotations, known drug resistance profiles, and the like. The specific pharmacological profiles include data on efficacy, toxicity, and side effects of therapeutic agents, enabling informed decision-making in clinical and preclinical settings. The target disease pathways highlight critical biochemical routes involved in disease progression, assisting in the identification of key intervention points. The mechanism of action data provides insights into how drugs interact with a biological target, improving the precision of drug construction. The bioactivity annotations compile experimental and computational findings regarding the effects of compounds on specific biological systems. The known drug resistance profiles assist in developing plans to overcome resistance mechanisms, particularly in cancer therapy and infectious disease treatment.

In an exemplary embodiment, the generative molecule devising subsystem 208 is configured with the one or more ML models. The one or more ML models are configured to generate one or more molecular structures tailored for drug discovery and therapeutic applications. The one or more ML models are configured to perform, but not constricted to, at least one of: binding affinity optimization, structural conformity and feasibility, chemical space exploration, iterative feedback loop, and the like, based on the obtained at least one of: biological target data and therapeutic information. The generative molecule devising subsystem 208 analyses protein structures, ligand interactions, and biomolecular networks, allowing the system 102 to identify potential one or more drug like molecules with improved efficacy. The generative molecule devising subsystem 208 explores diverse chemical spaces, prioritizing unexplored regions with high potential for the one or more drug like molecules. By incorporating the iterative feedback loop, the one or more ML models refine the one or more molecular structures in real-time, ensuring that the one or more molecular structures align with desired pharmacological properties and target disease pathways.

The generative molecule devising subsystem 208 employs one of: one or more structure-based approaches and ligand-based procedures to generate a pharmacophore-based configuration associated with the one or more molecular structures that facilitate molecular interaction analysis. The one or more structure-based approaches employ three-dimensional (3D) protein structures to construct the one or more molecules structures that fit precisely within target binding sites, whereas the ligand-based procedures rely on known bioactive molecules to generate similar yet optimized structures. One of: the one or more structure-based approaches and the ligand-based procedures approaches enable the system 102 to analyse one or more molecular graphs, evaluate chemical compositions, and generate precise 3D conformers that closely mimic naturally occurring interactions. The one or more ML models may include one or more transformer-based architectures with attention mechanisms tailored to recognize patterns within the one or more molecular graphs, enabling precise 3D conformer generation. The incorporation of at least one of: binding site analysis and structure-based design principles ensures that generated one or more molecular structures exhibit high binding specificity and enhanced pharmacological potential. At least one of: the binding site analysis and the structure-based design principles optimise binding interactions with the biological target. By refining molecular geometry and chemical properties, the system 102 significantly enhances the likelihood of discovering effective one or more drug like molecules with minimal side effects.

The generative molecule devising subsystem 208 ensures the structural conformity by leveraging the one or more ML models to generate the one or more molecular structures that precisely match a binding site geometry while maintaining synthetic feasibility. By analysing spatial configurations and molecular interactions, the one or more ML models refine structural properties to enhance binding affinity and pharmacological effectiveness. This involves evaluating geometric constraints, steric hindrances, and chemical compatibility to ensure that the one or more molecular structures fit optimally within the biological target. Additionally, the system 102 integrates synthetic feasibility checks, ensuring that the generated one or more molecular structures may be practically synthesized using available chemical methodologies.

The one or more ML models may comprise, but not restricted to, at least one of: one or more reinforcement learning-based (RL) models, the one or more transformer-based architectures, one or more protein and chemical language models, one or more Large Language Models (LLMs), one or more Small Large Language Models (SLLMs), one or more Graphics Processing Unit (GPU)-based models, one or more quantum models, and the like, to generate the one or more molecular structures with the optimized binding affinity. The one or more RL models iteratively improve molecular structure generation by rewarding high-affinity and synthetically feasible structures, while the one or more transformer-based architectures leverage deep learning techniques for precise sequence and molecular pattern recognition. By employing the deep learning techniques, including the one or more transformer-based architectures, the system 102 encodes reaction information, predicting retrosynthesis steps based on the one or more molecular structures and reactivity patterns.

Within the one or more transformer-based architectures, one or more self-attention mechanisms allow the system 102 to weigh different aspects of the one or more molecular structures and prioritize essential reaction centres, refining the predictions for both feasible starting materials and intermediate products in multi-step synthesis. By integrating the one or more RL models and sampling, the generative molecule devising subsystem 208 avoids redundant regions and identifies unique structures with high potential. The one or more protein and chemical language models employ vast biochemical datasets to generate the one or more molecular structures with the optimized binding affinity and enhanced drug-like properties.

The one or more LLMs are trained on vast datasets to understand and generate complex one or more molecular structures. The one or more SLLMs are optimized, lightweight versions of the one or more LLMs employed for efficient molecular analysis with reduced computational overhead. The one or more GPU-based models leverage parallel computing capabilities of the GPUs to accelerate high-throughput molecular simulations and drug discovery tasks. The one or more quantum models utilize Quantum Machine Learning (QML) principles to exploit quantum superposition and entanglement for enhanced molecular property predictions, chemical reaction simulations, and drug-target interaction assessments.

In an exemplary embodiment, the virtual screening subsystem 210 is configured to evaluate the generated one or more molecular structures. This evaluation focuses on critical aspects such as, but not limited to, at least one of: binding affinity, pharmacological relevance, structural validity, and the like, ensuring that only the most capable one or more molecular structures are considered for further development. The virtual screening subsystem 210 is configured with one or more virtual screening models to compute multi-parametric scoring. The multi-parametric scoring may comprise, but not restricted to, at least one of: docking score, ligand efficiency score, pharmacophore match score, and the like. The virtual screening subsystem 210 is configured to rank the generated one or more molecular structures based on the multi-parametric scoring. This ranking process ensures that the most effective and synthetically viable one or more molecular structures are prioritized for further investigation. An optimal docking score indicates a stronger binding affinity, while an optimal ligand efficiency score suggests an optimal balance between molecular weight and binding strength. Similarly, an optimal pharmacophore match score confirms the alignment of molecular features with essential pharmacological interactions. The one or more virtual screening models may comprise, but not limited to, at least one of: computational docking models, quantitative structure-activity relationship (QSAR) models, and the like. The computational docking models are configured to simulate the interaction between a molecular structure of the one or more molecular structures and the biological target, estimating binding strength. The QSAR models are configured to analyse molecular properties to predict bioactivity and therapeutic potential. The virtual screening subsystem 210 provides a comprehensive assessment of each generated molecular structure of the one or more molecular structures.

In an exemplary embodiment, the prediction subsystem 212 is configured to predict one or more properties associated with absorption, distribution, metabolism, excretion, and toxicity (ADMET) of the evaluated one or more molecular structures. The prediction subsystem 212 utilizes the one or more generative AI models trained on diverse datasets. The one or more generative AI models are also pre-trained on extensive datasets comprising protein-ligand interactions, ligand conformers, binding affinities, and pharmacokinetic profiles. Fine-tuning is performed on specific target binding sites using structural data and interaction fingerprints. By analysing the one or more molecular structures, the one or more generative AI models estimate the one or more properties, ensuring that only the one or more drug like molecules with favourable characteristics are selected. The one or more properties may comprise, but not constricted to, at least one of: solubility, permeability, metabolic stability, potential toxicity, and the like. The solubility affects drug bioavailability. The permeability influences absorption efficiency. The metabolic stability determines how long a drug remains active in a body of the user. The potential toxicity assesses adverse effects on the biological systems.

The one or more generative AI models are one or more graph neural network (GNN)-based models. The training of the one or more GNN-based models on diverse datasets enables accurate predictions by capturing complex molecular interactions and structural relationships. The diverse datasets may include, but not constrained to, at least one of: the pharmacokinetic data, the toxicology data, and the like, to predict essential drug-like properties. The drug-like properties refer to a set of physicochemical and pharmacokinetic characteristics that determine whether a molecule is configured with the potential to be an effective and safe drug like molecule of the one or more drug like molecules. The drug-like properties include molecular weight, lipophilicity (Log P), solubility, hydrogen bond donors and acceptors, polar surface area, and synthetic accessibility. Additionally, drug-like properties encompass ADMET (Absorption, Distribution, Metabolism, Excretion, and Toxicity) characteristics, ensuring that the generated molecules exhibit favourable bioavailability, minimal toxicity, and appropriate metabolic stability. The pharmacokinetic data enables the prediction subsystem 212 to understand how the one or more molecular structures behave in the biological systems and the toxicology data assesses safety concerns. This approach streamlines drug like molecule selection, optimizing molecular properties for clinical success while minimizing experimental costs and development timelines.

In an exemplary embodiment, the retrosynthesis planning subsystem 214 is configured to determine one or more feasible synthesis pathways for the selected one or more molecular structures using the one or more retrosynthesis models. The one or more retrosynthesis models at least one of: analyse reaction pathways and integrate information from one or more chemical reaction databases associated with the one or more databases 116 to generate the one or more feasible synthesis pathways. Reaction informatics enables the one or more retrosynthesis models to identify at least one of: optimal reaction conditions, experimental procedures, and impurity and yield predictions, ensuring practical and efficient lab implementations. The reaction informatics refers to the use of computational techniques to analyse the reaction pathways, predict the one or more feasible synthesis pathways, and integrate the one or more chemical reaction databases for retrosynthesis planning. The one or more retrosynthesis models may comprise, but not limited to, at least one of: one or more reaction template-based models, one or more transformer-based retrosynthesis models, and the like. By leveraging at least one of: the one or more reaction template-based models and the one or more transformer-based retrosynthesis models, the retrosynthesis planning subsystem 214 ensures that the identified one or more feasible synthesis pathways align with well-established reaction mechanisms. The retrosynthesis planning subsystem 214 systematically deconstructs complex molecules into simpler precursor structures, identifying viable intermediates and reagents needed for synthesis. This step is crucial in pharmaceutical and chemical research, as the system 102 provides an efficient way to devise synthetic approaches for novel compounds, reducing trial-and-error experimentation in laboratory settings.

To enhance accuracy and practicality, the one or more retrosynthesis models may employ, but not constricted to, at least one of: single-step reactions procedures, multi-step synthesis pathway procedures, and the like. The single-step reactions procedures allow for a focused analysis of key transformation reactions, while the multi-step synthesis pathway procedures provide a comprehensive blueprint for the sequential construction of the complex one or more molecular structures. The single-step reactions procedures employ a “Text-to-Text Transfer Transformer” framework for immediate transformations. The multi-step synthesis pathway procedures employ a Monte Carlo tree search model, efficiently breaking down complex one or more molecular structures into synthetically accessible intermediates. Additionally, the one or more retrosynthesis models incorporate the optimal reaction conditions, including temperature, catalysts, solvents, and reagent concentrations, ensuring high synthetic feasibility. By predicting reaction yields, the retrosynthesis planning subsystem 214 aids one or more chemists in selecting the most efficient and cost-effective one or more feasible synthesis pathways. This approach significantly accelerates the drug discovery process by enabling one or more researchers to evaluate multiple synthesis options quickly and determine the one or more feasible synthesis pathways for laboratory implementation. Along with reaction pathways, the retrosynthesis planning subsystem 214 provides critical reaction details, such as the optimal reaction conditions and yield predictions, to inform practical lab implementations.

Furthermore, the retrosynthesis planning subsystem 214 integrates vendor-supplied reagent availability data to a streamline synthesis planning. This integration ensures that the one or more feasible synthesis pathways are not only theoretically viable but also practical based on real-world reagent accessibility. By considering commercially available starting materials and intermediates, the system 102 minimizes the need for custom synthesis of rare and expensive reagents, optimizing both cost and efficiency. For the multi-step synthesis pathway procedures, the system 102 applies sequence-to-sequence techniques, allowing the system 102 to capture dependencies across sequential reactions in the one or more feasible synthesis pathways.

In an exemplary embodiment, the iterative refinement subsystem 216 is configured to optimise the one or more molecular structures to generate the potential one or more drug like molecules with a validated synthetic feasibility and the drug-like properties. The iterative refinement subsystem 216 operates by continuously refining the one or more molecular structures using the one or more feedback obtained from at least one of: the generative molecule devising subsystem 208, the virtual screening subsystem 210, the prediction subsystem 212, and the retrosynthesis planning subsystem 214. The iterative refinement subsystem 216 ensures that the generated one or more molecular structures not only exhibit strong binding affinity to the biological target but also maintain favourable pharmacokinetic and pharmacodynamic properties. The one or more feedback allows for systematic improvements in molecular structure construction, leading to the identification of structurally viable and therapeutically relevant one or more drug like molecules. The one or more feedback of generation, screening, and refinement accelerates the drug like molecule optimization process and reduces dependence on manual intervention. By simulating binding and affinity at each iteration, the iterative refinement subsystem 216 refines the generated one or more molecular structures, eliminating low-affinity one or more drug like molecules early.

The iterative refinement subsystem 216 optimises the workflow to minimize iterations, reduce computational costs, and maximize success rates. The iterative refinement subsystem 216 employs pre-filtering stages such as Lipinski's Rule of Five and other medicinal chemistry filters to prioritize the one or more molecular structures. The iterative refinement subsystem 216 filters out undesirable one or more molecular structures based on toxicity and synthetic accessibility. The iterative refinement subsystem 216 filters the one or more drug like molecules for drug-likeness, bioavailability, toxicity, and other ADMET parameters. The iterative refinement subsystem 216 refines molecular structure selection based on predefined thresholds for safety and efficacy.

The iterative refinement subsystem 216 employs multi-objective optimization procedures to achieve a balance among at least one of: binding affinity, toxicity, synthetic accessibility, bioavailability, other related parameters, and the like in generating the one or more drug like molecules. The iterative refinement subsystem 216 fine-tunes the one or more molecular structures to enhance the therapeutic potential while ensuring that the one or more molecular structures are efficiently synthesized in laboratory conditions. The binding affinity is optimized to strengthen molecular interactions with the biological target, while the toxicity predictions are incorporated to minimize adverse effects. Additionally, the synthetic accessibility considerations ensure that the refined one or more molecular structures may be produced using commercially available reagents and the one or more feasible synthetic pathways.

The system 102 generates optimized one or more drug like molecules ready for experimental validation and wet-lab synthesis. The system 102 provides a ranked list of the one or more drug like molecules along with detailed one or more feasible synthesis pathways and predicted ADMET profiles.

In an exemplary embodiment, the data collection subsystem 218 is configured to collect curated datasets that serve as the foundation for drug discovery and molecular structure construction. The curated datasets include essential biological and chemical information such as, but not constricted to, at least one of: protein structures, chemical libraries, reaction pathways, absorption, distribution, metabolism, excretion, and toxicity (ADMET) profiles, and the like. The integration of the protein structures enables precise modelling of target-ligand interactions, while the chemical libraries provide a vast space for molecular structure exploration using distributed and parallelized architectures. Additionally, the reaction pathways support efficient synthesis planning, and the ADMET profiles contribute to early-stage drug safety and efficacy evaluations. The data collecting subsystem may utilize extensive datasets from the chemical libraries, binding affinity studies, and publicly available protein-ligand complexes (e.g., Protein Data Bank binding). Incorporates real-world binding data from proprietary and open-source repositories to enhance predictive accuracy.

In an exemplary embodiment, the data pre-processing subsystem 220 standardizes and refines the curated datasets into a defined format suitable for computational analysis. The data pre-processing subsystem 220 eliminates duplicate entries to prevent redundancy and inconsistencies within the curated datasets. Additionally, the data pre-processing subsystem 220 annotates relevant one or more molecular features, such as, but not limited to, at least one of: pharmacophores (specific structural components responsible for biological activity), binding affinities (indicate the strength of interactions between the one or more molecular structures and the biological targets), and the like.

In an exemplary embodiment, the model training subsystem 222 is configured to train at least one of: the one or more ML models and the one or more generative AI models on diverse chemical datasets to enable structure-based molecular representation, ligand-based molecule structures, and bioactivity-labelled datasets. This training ensures that at least one of: the one or more ML models and the one or more generative AI models learn essential chemical and biological properties. The one or more generative AI models are trained for physicochemical properties, bioavailability, and toxicity prediction. The structure-based molecular representations allow at least one of: the one or more ML models and the one or more generative AI models to recognize molecular conformations and the potential interactions with the biological target. The ligand-based molecule structures aid in generating the one or more molecular structures with the one or more properties. The bioactivity-labelled datasets provide crucial insights into molecular efficacy, toxicity, and pharmacological properties, enhancing the predictive capabilities of at least one of: the one or more ML models and the one or more generative AI models.

The model training subsystem 222 is configured to develop the one or more virtual screening models. The one or more virtual screening models are devised by incorporating at least one of: one or more ligand-protein interactions, quantitative structure-activity relationship (QSAR) analysis, docking procedures, and the like. The one or more ligand-protein interaction analyses the binding affinity between the one or more drug like molecules and the biological target, predicting potential efficacy. The QSAR analysis establishes quantitative correlations between the one or more molecular structures and the biological activity. The docking procedures simulate how the one or more drug like molecules interact with the biological target at an atomic level, refining the one or more drug like molecules based on the binding efficiency. The docking procedures predict ligand binding modes with the biological target. The trained one or more virtual screening models facilitate the rapid evaluation and ranking of the one or more molecular structures based on the pharmacological relevance and structural viability. The model training subsystem 222 integrates binding pocket analysis using spatial features derived from molecular docking results. The system 102 implements distributed GPU computing for large-scale molecular docking simulations, reducing computational time significantly. Modular workflows support simultaneous ligand docking across multiple protein conformations.

Furthermore, the model training subsystem 222 is configured to create the one or more retrosynthesis models that generate the one or more feasible synthesis pathways for the one or more drug like molecules. The model training subsystem 222 incorporate at least one of: reaction templates (predefined chemical reaction patterns that guide synthesis), text-based pathways (use natural language processing techniques to analyse and extract reaction sequences from literature and the one or more databases 116), and the like. The reaction templates provide a structured approach to retrosynthesis, enabling the generation of the one or more feasible synthesis pathways based on established chemical transformations. The text-based pathways allow retrosynthesis planning by extracting and interpreting chemical reaction data from scientific literature.

The system 102 is configured to link the plurality of subsystems 110 to ensure seamless data exchange between the one or more generative AI models, screening, synthesis, and prediction workflows. The system 102 employs orchestration frameworks for efficient parallel processing and multi-parametric optimization. The system 102 validates with benchmark datasets to test molecular structure generation, virtual screening, retrosynthesis accuracy, and ADMET predictions.

In an exemplary embodiment, as shown in FIG. 2B, the user inputs specific criteria with input (e.g., a Simplified Molecular Input Line Entry (SMILE) string, an input file, a target protein, generation filters). The one or more generative AI models take input as SMILE notation of one of: desired molecule, the target protein from a Protein Data Bank ID (PDB), a target protein structure (which produces the one or more molecular structures that meet the input specifications), and the like. The one or more structure-based approaches employ available protein structures and predicted protein structures using an AlphaFold. The AlphaFold is an AI model that predicts protein folding. If a protein structure is unavailable, alternative sequence-based predictions may be used. The ligand-based procedures focus on generating the one or more molecular structures based on known ligands, facilitating pharmacophore-based molecule generation. The system employs hit identification, which ranks and prioritizes the one or more molecular structures based on binding affinity, pharmacophore matching, and quantitative structure-activity relationship (QSAR) reward functions. The multi-parametric optimization ensures that the one or more selected molecular structures meet various criteria such as, but not constrained to, at least one of: MedChem filters, structural alerts, diversity, novelty, synthetic accessibility score (SAS), and quantitative estimate of drug-likeness (QED). The system applies descriptor filters to refine the potential one or more drug like molecules by assessing the physicochemical properties, the drug-likeness properties, and medicinal chemistry (MedChem) constraints.

The generated one or more molecular structures undergo ADMET property screening, filtering out the one or more drug like molecules with poor drug-like characteristics. The retrosynthesis planning subsystem 214 evaluates the reactants, reaction conditions, vendor information from the vendor-supplied reagent availability data, ensuring each drug like molecule of the one or more drug like molecules is configured with a feasible synthesis pathway of the one or more feasible synthesis pathways. High-affinity one or more molecular structures are selected through virtual screening, narrowing down to the one or more drug like molecules. By automating molecule construction, synthesis route prediction, and screening, the system 102 reduces time-to-lead by up to 50 percent. The automated construction and optimization of the one or more drug like molecules cut down laboratory and material costs. Early ADMET prediction and retrosynthesis reduce downstream failure rates. The system 102 enhances chemical diversity, creating innovative one or more drug like molecules that traditional methods may overlook.

In an exemplary embodiment, when an input a Simplified Molecular Input Line Entry (SMILE) string 1 is given to the system 102, the system determines the one or more properties of the SMILE string. The physico-chemical properties are derived from the input as shown in Table 1. The one or more properties associated with the absorption (shown in Table 2), the distribution (shown in Table 3), the metabolism (shown in Table 4), the excretion (shown in Table 5), and the toxicity (shown in Table 6) are computed by the system 102.

TABLE 1

| | | Ideal |
|---|---|---|
| Property | Value | Range |
| logP | 3.626 | 0-5 |
| Mol wt | 402.531 | 200-500 |
| nHA | 5 | 0-10 |
| nHD | 1 | 0-5 |
| Lipinski | 4 | 0-4 |
| QED | 0.714 | 0-1 |
| StereoCenters | 8 | 0- |
| | | infinite |
| HFE | −5.164 | (−Infinite)- |
| | | infinite |
| TPSA | 80.67 | 0- |
| | | infinite |

TABLE 2

| | | Ideal |
|---|---|---|
| Property | Value | Range |
| Caco-2 | −4.497 | (−Infinite)- |
| Permeability | | infinite |
| HIA | 1 | 0-1 |
| Oral Bio av | 0.781 | 0-1 |
| Lipophilicity | 3.44 | (−Infinite)- |
| | | infinite |
| Aqua Solubility | −5.234 | (−Infinite)- |
| | | infinite |
| Pgp Inhibitor | 0.797 | 0-1 |

TABLE 3

| | | Ideal |
|---|---|---|
| Property | Value | Range |
| PPB | 96.57 | 0-100 |
| Vol Dist | −5.987 | 0-infinite |
| PAMPA | 0.984 | 0-1 |
| BBB | 0.984 | 0-1 |

TABLE 4

| | | Ideal |
|---|---|---|
| Property | Value | Range |
| CYP1A2 | 0.004 | 0-1 |
| Inhibitor | | |
| CYP2C19 | 0.616 | 0-1 |
| Inhibitor | | |
| CYP2D6 | 0.047 | 0-1 |
| Inhibitor | | |
| CYP3A4 | 0.887 | 0-1 |
| Inhibitor | | |
| CYP2C9 | 0.405 | 0-1 |
| Inhibitor | | |
| CYP2D6 | 0.047 | 0-1 |
| Substrate | | |
| CYP3A4 | 0.438 | 0-1 |
| Inhibitors | | |
| CYP2C9 | 0.096 | 0-1 |
| Substrate | | |

TABLE 5

| | | Ideal |
|---|---|---|
| Property | Value | Range |
| Half-Life | −13.152 | 0- |
| | | infinite |
| Clearance | 97.055 | 0- |
| hepatocyte | | infinite |
| Clearance | 74.417 | 0- |
| Microsome | | infinite |

TABLE 6

| | | Ideal |
|---|---|---|
| Property | Value | Range |
| LD50 (Zhu) | 2.424 | 0- |
| | | infinite |
| hERG Blockers | 0.252 | 0-1 |
| Ames | 0.108 | 0-1 |
| Mutagenicity | | |
| DILI | 0.219 | 0-1 |
| Carcinogens | 0.067 | 0-1 |
| Clintox | 0.038 | 0-1 |
| NR-AR-LBD | 0.096 | 0-1 |
| NR-AR | 0.511 | 0-1 |
| NR-AhR | 0.034 | 0-1 |
| Nuclear | 0.092 | 0-1 |
| Aromatase | | |
| NR-ER-LBD | 0.019 | 0-1 |
| NR-ER | 0.165 | 0-1 |
| NR-PPAR- | 0.009 | 0-1 |
| gamma | | |
| SR-ARE | 0.374 | 0-1 |
| SR-ATAD5 | 0.008 | 0-1 |
| SR-HSE | 0.038 | 0-1 |
| SR-MMP | 0.468 | 0-1 |
| SR-P53 | 0.025 | 0-1 |
| Skin Rxn | 0.212 | 0-1 |

### SMILE String 1:

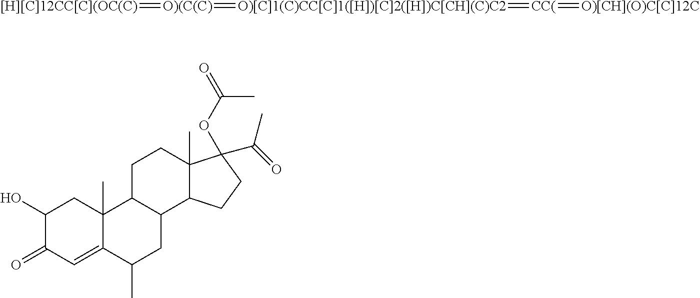

In another exemplary embodiment, for input SMILE string 2, using the single-step reactions procedures, the system 102 provides a reactant 1 and a reactant 2 (one or more drug like molecules) along with the one or more properties shown in Table 7. The one or more drug like molecules are displayed on a user interface associated with the one or more communication devices 114.

### SMILE String 2:

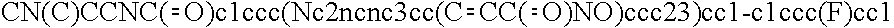

### Reactant 1:

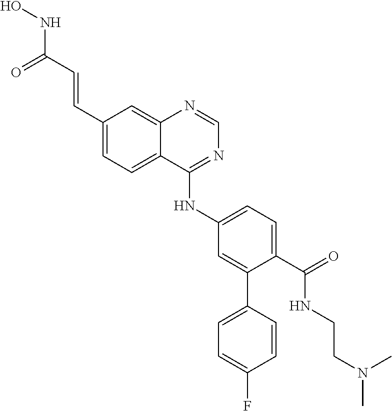 [SMILES: CN(C)CCNC(=O)c1ccc(Nc2ncnc3cc(/C=C/C(=O)NO)ccc23)cc1-c1ccc(F)cc1]

### Reactant 2:

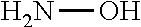

TABLE 7

| Property | Reactant 1 | Reactant 2 |
|---|---|---|
| molWeight | 499.5460000000002 | 33.03 |
| logp | 4.568700000000002 | −0.6657000000000002 |
| tpsa | 107.45 | 46.25 |
| hbd | 3 | 2 |
| hba | 6 | 2 |
| rotatableBonds | 9 | N/A |
| fractionSp3 | 0.14285714285714285 | N/A |
| numAtoms | 37 | 2 |
| numHeavyAtoms | 37 | 2 |
| numAromaticRings | 4 | N/A |
| numRings | 4 | N/A |

In another exemplary embodiment, for an input SMILE string 3, using the multi-step synthesis pathway procedures, the system 102 generates the output shown in FIG. 2C. The output is generated using the Monte Carlo tree search model. The displayed pathway in FIG. 2C represents a highest-confidence pathway among multiple predicted feasible synthesis pathways.

### SMILE String 3:

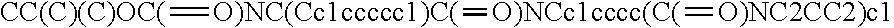

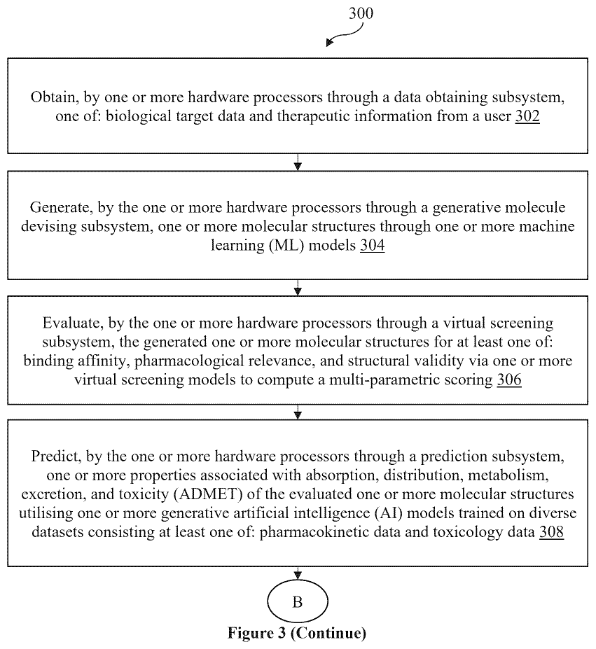

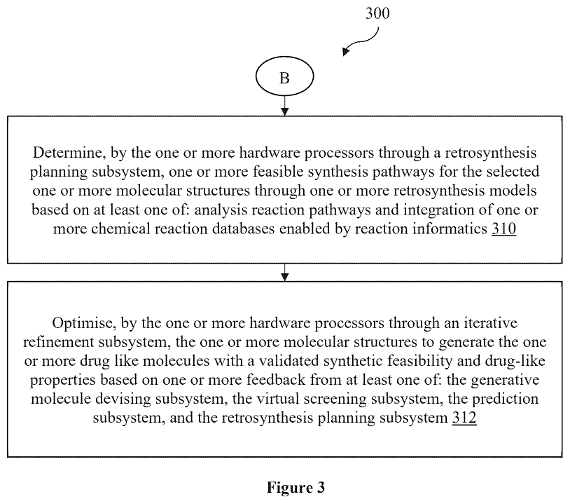

FIG. 3 illustrates an exemplary flow diagram depicting an artificial intelligence-based (AI-based) method 300 for generating the one or more drug like molecules, in accordance with an embodiment of the present disclosure.

According to an exemplary embodiment of the disclosure, the AI-based method 300 for generating the one or more drug like molecules is disclosed. At step 302, the AI-based method 300 includes the data-obtaining subsystem that acquires at least one of: the biological target data and the therapeutic information using the one or more hardware processors. The biological target data may comprise, but not restricted to, at least one of: protein structures, ligand data, gene expression profiles, biomolecular interaction networks, and disease-associated biomarkers. The therapeutic information may comprise, but not restricted to, at least one of: specific pharmacological profiles, target disease pathways, mechanism of action data, bioactivity annotations, and known drug resistance profiles.

At step 304, the AI-based method 300 includes the generative molecule devising subsystem that employs the one or more ML models to generate the one or more molecular structures based on at least one of: the biological target data and the therapeutic information. The one or more ML models are configured to optimise the binding affinity, ensuring strong interactions between the generated one or more molecular structures and the biological target. The one or more ML models maintain the structural conformity and feasibility by aligning the one or more molecular structures with the binding site geometry while ensuring synthetic accessibility. Additionally, the one or more ML models explore the chemical space and refine molecular constructions iteratively based on the iterative feedback loop from at least one of: the biological target data and the therapeutic information.

At step 306, the AI-based method 300 includes the virtual screening subsystem that assesses the generated one or more molecular structures for at least one of: the binding affinity, the pharmacological relevance, and the structural validity. The virtual screening subsystem utilizes the one or more virtual screening models to evaluate the molecular interactions. The multi-parametric scoring approach ensures accurate ranking of the potential one or more drug like molecules. The multi-parametric scoring may comprise, but not limited to, at least one of: the docking score, the ligand efficiency score, and the pharmacophore match score. The one or more virtual screening models may comprise, but not limited to, at least one of: the computational docking models and the QSAR models.

At step 308, the AI-based method 300 includes the prediction subsystem configured with the one or more generative AI models to predict the one or more properties, ensuring drug-like characteristics of the one or more molecular structures. The one or more generative AI models are trained on the diverse datasets. The diverse datasets may comprise, but not constricted to, at least one of: the pharmacokinetic data and the toxicology data. The one or more properties may comprise, but not constricted to, at least one of: the solubility, the permeability, the metabolic stability, and the potential toxicity. The one or more generative AI models are the one or more GNN-based models. By leveraging the one or more GNN-based models, the prediction subsystem enhances the accuracy of ADMET predictions, aiding in the identification of viable one or more drug like molecules.

At step 310, the AI-based method 300 includes the retrosynthesis planning subsystem that determines the one or more feasible synthesis pathways for the selected one or more molecular structures by at least one of: analysing the reaction pathways and integrating the one or more chemical reaction databases. The retrosynthesis planning subsystem employs the one or more retrosynthesis models enabled by the reaction informatics, to generate the optimal one or more feasible synthesis pathways. The one or more retrosynthesis models may comprise, but not constrained to, at least one of: the one or more reaction template-based models and the one or more transformer-based retrosynthesis models.

At step 312, the AI-based method 300 includes the iterative refinement subsystem that optimizes the one or more molecular structures to generate the one or more drug like molecules with the validated synthetic feasibility and the drug-like properties. The iterative refinement subsystem incorporates the one or more feedback from the generative molecule devising subsystem, the virtual screening subsystem, the prediction subsystem, and the retrosynthesis planning subsystem. By employing the multi-objective optimization, the iterative refinement subsystem balances at least one of: the binding affinity, the toxicity, the synthetic accessibility, and the bioavailability to refine the one or more molecular structures effectively.

The AI-based method 300 further includes the data collection subsystem 218 that collects the curated datasets. The curated datasets are configured to drive the drug discovery process. By collecting the curated datasets, the data collection subsystem 218 ensures that the information used for constructing and optimizing the one or more molecular structures is comprehensive and well-rounded. The curated datasets may comprise, but not constrained to, at least one of: the protein structures, the chemical libraries, the reaction pathways, and the ADMET profiles.

The AI-based method 300 further includes the data pre-processing subsystem that pre-processes the curated datasets into the defined format to ensure consistency. This involves tasks such as eliminating duplicate entries to maintain data integrity and annotating the relevant one or more features such as, but not restricted to, at least one of: the pharmacophores and the binding affinities.

The AI-based method 300 further includes the model training subsystem that leverages the diverse chemical datasets to train at least one of: the one or more ML and the one or more generative AI models, focusing on at least one of: the structure-based molecule representations, the ligand-based molecule structures, and the bioactivity-labelled datasets. By training on the diverse chemical datasets, the model training subsystem equips at least one of: the one or more ML and the one or more generative AI models to understand complex molecular interactions and predict bioactivity with high accuracy.

The AI-based method 300 further includes the model training subsystem that devises the one or more virtual screening models by leveraging at least one of: the one or more ligand-protein interactions, the QSAR analysis, and the docking procedures. The one or more virtual screening models are trained to assess and predict the binding affinity and effectiveness of the one or more molecular structures against the biological target.

The AI-based method 300 further includes the model training subsystem that creates the one or more retrosynthesis models by incorporating at least one of: the reaction templates and the text-based pathways. The one or more retrosynthesis models are configured to predict feasible chemical reactions for the synthesis of the target one or more molecular structures.

Numerous advantages of the present disclosure may be apparent from the discussion above. In accordance with the present disclosure, the system for generating the one or more drug like molecules is disclosed. The system streamlines molecule construction, optimizes the one or more drug like molecules, predicts the one or more feasible synthesis pathways, and evaluates the drug-like properties with unparalleled efficiency. By automating key components of a Design-Make-Test-Analyse (DMTA) cycle, the system significantly reduces the number of iterations required, thereby minimizing time, cost, and dependency on experimental resources. The modular yet interconnected workflows of the system enable seamless scalability, making the system adaptable for a wide range of therapeutic areas. The system represents a paradigm shift in computer-aided drug discovery, addressing critical bottlenecks in the process and accelerating the journey from concept to clinic. The system evaluates routes for commercial viability, reaction conditions, and cost-effectiveness. The system provides an end-to-end automation of construction, screening, and synthesis.

The system reduces the iterations in the DMTA cycle, cutting time and costs significantly. The system supports diverse therapeutic targets and the chemical spaces. The system provides real-time insights into the molecular properties and synthesis feasibility. supporting large-scale molecule assessments. The system enables efficient evaluation of extensive compound libraries, accelerating early-stage filtering. The system predicts and refines pharmacokinetic attributes pre-experimentally, aligning the molecule properties with therapeutic objectives. The system focuses on the one or more molecular structures with favourable ADMET profiles, reducing late-stage attrition risks. The system selects the one or more feasible synthesis pathways that maximize synthesis efficiency while reducing unnecessary steps. By simulating both the single-step reactions procedures and the multi-step synthesis pathway procedures, the system allows for rapid generation of multiple synthesis options, optimizing for both feasibility and cost. The system is configured with integrated reaction conditions and yield predictions that provide lab-ready synthesis guidance, reducing experimental trial-and-error. The attention to reaction specificity and sequence dependencies enables more precise synthetic plans. The access to the vendor-supplied reagent availability data for reagent sourcing ensures that the synthesis plans align with available materials, to avoid procurement delays.

The system handles billion-scale compound libraries with minimal computational overhead through distributed workflows. The system delivers rapid predictions, accelerating the DMTA cycle in drug discovery. The system improves hit identification rates and mitigates false positives through integrated post-screening validation. The system adapts to diverse targets and disease areas with flexible scoring functions and parameterization options. The system supports user-defined compound libraries, scoring schemes, and filtering criteria. The system reduces the need for experimental high-throughput screening by identifying the optimal one or more drug like molecules computationally. The system saves resources by focusing only on promising compounds for experimental validation and also provides intuitive visualizations and interpretive tools, empowering medicinal chemists and researchers to make informed decisions. A modular and scalable configuration of the system ensures accessibility for academic institutions, startups, and mid-sized biotech firms, breaking the cost barriers associated with proprietary systems. The system supports interoperability with external databases, software, and tools, enabling seamless integration into existing research ecosystems. The system provides flexible customization options that allow the one or more researchers to configure workflows to suit specific drug discovery objectives. This accelerates drug discovery timelines by up to 25 percent and cuts costs by 60 percent. The system handles complex datasets and supports diverse drug discovery objectives, including rare diseases and large-scale projects. The system allows the user to tailor workflows for specific needs. Template- and template-free retrosynthesis are integrated with the system.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

I/We claim:

1. An artificial intelligence-based (AI-based) method for generating one or more drug like molecules, comprising:
obtaining, by one or more hardware processors through a data-obtaining subsystem, one of: biological target data and therapeutic information from a user;
generating, by the one or more hardware processors through a generative molecule devising subsystem, one or more molecular structures through one or more machine learning (ML) models,
the one or more machine learning (ML) models configured to perform at least one of: binding affinity optimization, structural conformity and feasibility, chemical space exploration, and iterative feedback loop based on the obtained one of: biological target data and therapeutic information;
evaluating, by the one or more hardware processors through a virtual screening subsystem, the generated one or more molecular structures for at least one of: binding affinity, pharmacological relevance, and structural validity via one or more virtual screening models to compute a multi-parametric scoring;
predicting, by the one or more hardware processors through a prediction subsystem, one or more properties associated with absorption, distribution, metabolism, excretion, and toxicity (ADMET) of the evaluated one or more molecular structures utilising one or more generative artificial intelligence (AI) models trained on diverse datasets consisting at least one of: pharmacokinetic data and toxicology data;
determining, by the one or more hardware processors through a retrosynthesis planning subsystem, one or more feasible synthesis pathways for the selected one or more molecular structures through one or more retrosynthesis models based on at least one of: analysis reaction pathways and integration of one or more chemical reaction databases enabled by reaction informatics; and
optimising, by the one or more hardware processors through an iterative refinement subsystem, the one or more molecular structures to generate the one or more drug like molecules with a validated synthetic feasibility and drug-like properties based on one or more feedback from at least one of: the generative molecule devising subsystem, the virtual screening subsystem, the prediction subsystem, and the retrosynthesis planning subsystem.

2. The artificial intelligence-based (AI-based) method as claimed in claim 1, comprising:
collecting, by the one or more hardware processors through a data collection subsystem, curated datasets comprise at least one of: protein structures, chemical libraries, reaction pathways, and absorption, distribution, metabolism, excretion, and toxicity (ADMET) profiles;
pre-processing, by the one or more hardware processors through a data pre-processing subsystem, the curated datasets into a defined format by performing at least one of: eliminating duplicates and annotate relevant one or more features comprise at least one of: pharmacophores and binding affinities;
training, by the one or more hardware processors through a model training subsystem, at least one of: the one or more machine learning (ML) models and the one or more generative artificial intelligence (AI) models, on diverse chemical datasets for structure-based molecule representations, ligand-based molecule structures, and bioactivity-labelled datasets;
devising, by the one or more hardware processors through the model training subsystem, the one or more virtual screening models based on leveraging at least one of: one or more ligand-protein interactions, quantitative structure-activity relationship (QSAR) analysis, and docking procedures; and
creating, by the one or more hardware processors through the model training subsystem, the one or more retrosynthesis models by incorporating at least one of: reaction templates and text-based pathways.

3. The artificial intelligence-based (AI-based) method as claimed in claim 1, wherein
the biological target data comprises at least one of: protein structures, ligand data, gene expression profiles, biomolecular interaction networks, and disease-associated biomarkers; and
the therapeutic information comprises at least one of: specific pharmacological profiles, target disease pathways, mechanism of action data, bioactivity annotations, and known drug resistance profiles.

4. The artificial intelligence-based (AI-based) method as claimed in claim 1, wherein
employing, by the one or more machine learning (ML) models, one of: one or more structure-based approaches and ligand-based procedures to generate a pharmacophore-based configuration associated with the one or more molecular structures to analyse at least one of: one or more molecular graphs and generate precise three dimensional (3D) conformers;
generating, by the one or more machine learning (ML) models, the one or more molecular structures based on incorporating at least one of: a binding site analysis and structure-based design principles, to optimize binding interactions with a biological target; and
providing, by the one or more machine learning (ML) models, a structural conformity by generating the one or more molecular structures matches with a binding site geometry and maintaining synthetic feasibility,
the one or more machine learning (ML) models comprise at least one of: one or more reinforcement learning-based (RL) models, one or more transformer-based architectures, one or more protein and chemical language models, one or more Large Language Models (LLMs), one or more Small Large Language Models (SLLMs), one or more Graphics Processing Unit (GPU)-based models, and one or more quantum models, to generate the one or more molecular structures with optimized binding affinity.

5. The artificial intelligence-based (AI-based) method as claimed in claim 1, wherein ranking, by the virtual screening subsystem, the generated one or more molecular structures based on the multi-parametric scoring,
the multi-parametric scoring comprises at least one of: docking score, ligand efficiency score, and pharmacophore match score.

6. The artificial intelligence-based (AI-based) method as claimed in claim 1, wherein the one or more virtual screening models comprise at least one of: computational docking models and quantitative structure-activity relationship (QSAR) models.

7. The artificial intelligence-based (AI-based) method as claimed in claim 1, wherein the one or more properties comprise at least one of: solubility, permeability, metabolic stability, and potential toxicity.

8. The artificial intelligence-based (AI-based) method as claimed in claim 1, wherein the one or more generative artificial intelligence (AI) models are one or more graph neural network (GNN)-based models,
training the one or more graph neural network (GNN)-based models on the diverse datasets including at least one of: the pharmacokinetic data and the toxicology data to predict essential drug-like properties.

9. The artificial intelligence-based (AI-based) method as claimed in claim 1, wherein the one or more retrosynthesis models comprise at least one of: one or more reaction template-based models and one or more transformer-based retrosynthesis models,
the one or more retrosynthesis models employ at least one of: single-step reactions procedures and multi-step synthesis pathway procedures to determine the one or more feasible synthesis pathways along with at least one of: optimal reaction conditions experimental procedures, impurity and yield predictions to report practical lab implementations.

10. The artificial intelligence-based (AI-based) method as claimed in claim 1, wherein
generating, by the retrosynthesis planning subsystem, the one or more feasible synthesis pathways by integrating vendor-supplied reagent availability data to provide a streamlined synthesis planning.

11. The artificial intelligence-based (AI-based) method as claimed in claim 1, wherein
incorporating, by the iterative refinement subsystem, multi-objective optimization procedures to balance at least one of: binding affinity, toxicity, synthetic accessibility, and bioavailability, in generating the one or more drug like molecules.

12. An artificial intelligence-based (AI-based) system for generating one or more drug like molecules, comprising:
one or more servers, comprising:
one or more hardware processors; and
a memory unit coupled to the one or more hardware processors, wherein the memory unit comprises a plurality of subsystems in form of one or more instructions executable by the one or more hardware processors, and wherein the plurality of subsystems comprises:
a data-obtaining subsystem configured to obtain one of: biological target data and therapeutic information from a user;
a generative molecule devising subsystem configured to generate one or more molecular structures through one or more machine learning (ML) models,
the one or more machine learning (ML) models configured to perform at least one of: binding affinity optimization, structural conformity and feasibility, chemical space exploration, and iterative feedback loop based on the obtained one of: biological target data and therapeutic information;
a virtual screening subsystem configured to evaluate the generated one or more molecular structures for at least one of: binding affinity, pharmacological relevance, and structural validity via one or more virtual screening models to compute a multi-parametric scoring;
a prediction subsystem configured to predict one or more properties associated with absorption, distribution, metabolism, excretion, and toxicity (ADMET) of the evaluated one or more molecular structures by one or more generative artificial intelligence (AI) models trained on diverse datasets consisting at least one of: pharmacokinetic data and toxicology data;
a retrosynthesis planning subsystem configured to determine one or more feasible synthesis pathways for the selected one or more molecular structures through one or more retrosynthesis models based on at least one of: analysing reaction pathways and integration of one or more chemical reaction databases enabled by reaction informatics; and
an iterative refinement subsystem configured to optimise the one or more molecular structures for generating the one or more drug like molecules with a validated synthetic feasibility and drug-like properties based on one or more feedback from at least one of: the generative molecule devising subsystem, the virtual screening subsystem, the prediction subsystem, and the retrosynthesis planning subsystem.

13. The artificial intelligence-based (AI-based) system as claimed in claim 12, comprising:
a data collection subsystem configured to collect curated datasets comprise at least one of: protein structures, chemical libraries, reaction pathways, and absorption, distribution, metabolism, excretion, and toxicity (ADMET) profiles;
a data pre-processing subsystem configured to pre-process the curated datasets into a defined format by performing at least one of: eliminating duplicates and annotate relevant one or more features comprise at least one of: pharmacophores and binding affinities; and
a model training subsystem configured to:
train at least one of: the one or more machine learning (ML) models and the one or more generative artificial intelligence (AI) models, on diverse chemical datasets for structure-based molecule representations, ligand-based molecule structures, and bioactivity-labelled datasets;
devise the one or more virtual screening models based on leveraging at least one of: one or more ligand-protein interactions, quantitative structure-activity relationship (QSAR) analysis, and docking procedures; and
create the one or more retrosynthesis models by incorporating at least one of: reaction templates and text-based pathways.

14. The artificial intelligence-based (AI-based) system as claimed in claim 12, wherein one or more machine learning (ML) models configured to:
employ one of: one or more structure-based approaches and ligand-based procedures to generate a pharmacophore-based configuration associated with the one or more molecular structures to analyse at least one of: one or more molecular graphs and generate precise three dimensional (3D) conformers;
generate the one or more molecular structures based on incorporating at least one of: a binding site analysis and structure-based design principles, to optimize binding interactions with a biological target; and
provide a structural conformity by generating the one or more molecular structures matches with a binding site geometry and maintaining synthetic feasibility,
the one or more machine learning (ML) models comprise at least one of: one or more reinforcement learning-based (RL) models, one or more transformer-based architectures, one or more protein and chemical language models, one or more Large Language Models (LLMs), one or more Small Large Language Models (SLLMs), one or more Graphics Processing Unit (GPU)-based models, and one or more quantum models, to generate the one or more molecular structures with optimized binding affinity.
